(19) United States
(12) Patent Application Publication
(10) Pub. No.: US 2003/0144734 A1
(43) Pub. Date: Jul. 31, 2003

(54) Breast implant with locatable injection site

(76) Inventors: Paul A. Dreschnack, New Orleans, LA (US); Orterio S. III Villa, Slidell, LA (US)

Correspondence Address: Michael D. Carbo, Esq., Michael D. Carbo, P.L.C., Suite 700, 228 St. Charles Avenue, New Orleans, LA, 70130, US

(21) Appl. No.: 10/172,293

(22) Filed: Jun. 13, 2002

Related U.S. Application Data
(60) Provisional application No. 60/352,050, filed on Jan. 28, 2002.

(30) Foreign Application Priority Data
Mar. 11, 2002 (US) 10/095241

(51) Int. Cl. A61F 2/12

(52) U.S. Cl. 623/8; 623/912

(57) ABSTRACT

An implantable breast tissue prosthesis assembly comprising an envelope having a shell defining an inner fluid containable chamber and an outer surface, a valve, formed as part of a wall in the envelope, for facilitating the introduction, containment, or removal of fluid within the chamber, and a backing plate comprising material detectable by ultrasound detection technology and shaped to guide a needle into the valve.

### CROSS-REFERENCE TO RELATED APPLICATIONS

This patent claims priority of U.S. patent application Ser. No. 10/095,241, filed Mar. 11, 2002, entitled Natur Saline Filled Mammary Implant, and of U.S. patent application Ser. No. 60/352,050, filed Jan. 28, 2002, entitled Natur Saline Filled Breast Implants.

### STATEMENT REGARDING FEDERALLY SPONSORED RESEARCH OR DEVELOPMENT

Not applicable.

### BACKGROUND OF THE INVENTION

1. Field of the Invention

The invention relates to medical devices and methods. In particular, the present invention relates to an improved implantable breast prostheses having a non-magnetic, locatable injection site located integrally on an envelope of the prosthesis. The present invention also relates to methods for locating a port or valve used for filling a breast implant with saline or removing saline from the implant.

2. Description of Background Art

Implantable breast prostheses are used to augment the size or to modify the shape of a breast. Implantable prosthetic devices have been used throughout the body. The most common use has been for restoring or improving upon normal body contour or augmenting as well as reconstructing the female breast. Past attempts at breast modification have used discrete implants, usually a single chamber implant filled with a silicone product. (See U.S. Pat. Nos. 3,665,520; 3,681,787; 4,455,691; 4,573,993; 4,772,284). These past attempts often did not present a normal contour to a modified breast, and relatively large incisions were required for insertion.

Other prostheses have used multiple chambers (See U.S. Pat. No. 4,507,810 with multiple interconnecting chambers), but these implants often fail to provide a normal contour to the reconstructed or augmented breast, and they cannot typically be adjusted for the individual recipient. They also typically require large incisions.

Recently, separate “stacked” implants have been used in reconstruction or augmentation procedures, but they tend to drift apart or separate. Others use two immutably and angularly attached envelopes. Once bonded together, one conventional prosthesis is inserted into a cavity specifically formed for implantation purposes. Each envelope is filled with a fluid, such as saline, to achieve a desired breast form. See U.S. patent application Ser. No. 20,020,038,147 A1, which discloses valves, similar to ball valves, that are inserted into each of two envelopes that are bonded together.

It is known that when a breast prosthesis is implanted in the body, fibrous scar tissue encapsulates the prosthesis and leads to spherical scar contracture, which causes the breast prosthesis to change to an undesired shape. Two of the many ways to limit scar contracture include (1) overfilling the prosthesis at the time of implantation and subsequently bleeding off the excess fluid to allow for a softer implant, and (2) underfilling the implant at the time of implantation and subsequently adding fluid to counteract the forces of scar contracture with perhaps later bleed off. Sometimes a breast implant patient desires to change the size and contour of her breast.

Surgeons commonly use tissue expanders that are periodically inflated, usually with a sterile isotonic solution. These inflations usually occur in a sterile environment using a small gauge needle that is placed percutaneously through the skin, past a resealing septum, and into the tissue expander. Tissue expanders are intended to be removed upon completion of expansion. U. S. patent application Ser. No. 2001004709 A1 discloses a tissue expander having a magnetically locatable injection site designed into a hard-reinforced insert. Injection sites on tissue expanders are metal. But metal is disfavored for use in breast implants because magnetic resonance imaging (MRI) screening for breast cancer cannot be performed in the presence of metal.

Also, filling a breast implant with saline and bleeding of saline from a breast implant requires a surgical incision unless the filling/bleeding port is reliably locatable so that a needle puncture through the skin and the injection site can be used for filling/bleeding. The present invention overcomes the problems described above by providing an improved breast prosthesis comprised of a single-lumen or a double-lumen prosthesis having an injection port comprising a valve and having a nonmetallic backing plate that can be imaged by ultrasonic imaging equipment.

### BRIEF SUMMARY OF THE INVENTION

It is to be understood that both the foregoing general description and the following detailed description are exemplary and explanatory only, and are not restrictive of the invention as claimed. The accompanying drawings, which are incorporated herein by reference, and which constitute a part of this specification, illustrate certain embodiments of the invention and, together with the detailed description, serve to explain the principles of the present invention.

In this respect, before explaining at least one embodiment of the invention in detail, it is to be understood that the invention is not limited in this application to the details of construction and to the arrangement so the components set forth in the following description or illustrated in the drawings. The invention is capable of other embodiments and of being practiced and carried out in various ways. Also, it is to the understood that the phraseology and terminology employed herein are for the purpose of description and should not be regarded as limiting. As such, those skilled in the art will appreciate that the conception, upon which this disclosure is based, may readily be utilized as a basis for the designing of other structures, methods and systems for carrying out the several purposes of the present invention. It is important, therefore that the claims be regarded as including such equivalent constructions insofar as they do not depart from the spirit and scope of the present invention.

Further, the purpose of the foregoing abstract is to enable the U.S. Patent and Trademark Office and the public generally, and especially the design engineers and practitioners in the art who are not familiar with patent or legal terms or phraseology, to determine quickly from a cursory inspection the nature and essence of the technical disclosure of the application. The abstract is neither intended to define the invention of the application, which is measured by the claims, nor is it intended to be limiting as to the scope of the invention in any way.

It is an object of the present invention to provide a breast implant having integrated thereon a valve that will reduce the risk of leakage associated with valve failures.

It is another object of the invention to provide a breast implant having an integrated valve containing no magnetic materials.

It is a further object of the invention to provide a backing plate for the integrated valve that reduces the risk of iatrogenic puncturing of the breast implant envelope.

It is also an object of this invention to provide an implant that may be individualized or customized by the surgeon for each patient.

It is another object of this invention to provide an implant that can be adjusted in an office procedure postoperatively.

Yet another object of this invention is to reduce the need for a tissue expander in a mastectomized patient.

It is yet a further object of this invention to provide a method for making implants and for using implants in the human breast.

Another object of this invention is to provide a method for imaging nonmetallic valves on breast implants.

These objects are meant to be illustrative and not limiting. The manner of operation, novel features, and further objects and advantages of this invention may be better understood by reference to the accompanying drawing, description, and claims,

### BRIEF DESCRIPTION OF THE DRAWINGS

FIG. 1 is a front view of a single-chambered breast implant having one injection port for adjusting and/or resizing the implant after the implant is in place.

FIG. 2 is a front view of a two-chambered breast implant having an injection port on each chamber.

FIG. 3 is a side view of an embodiment of a backing plate according to the invention.

FIG. 4 is a top view of an embodiment of a backing plate.

FIG. 5 is a section view, taken along cut-line A-A, of an embodiment of a backing plate.

FIG. 6 is a profile view of an embodiment of a backing plate.

### DETAILED DESCRIPTION OF THE INVENTION

While the making and using of various embodiments of the present invention are discussed in detail below, it should be appreciated that the present invention provides for inventive concepts capable of being embodied in a variety of specific contexts. The specific embodiments discussed herein are merely illustrative of specific manners in which to make and use the invention and are not to be interpreted as limiting the scope of the instant invention.

The claims and the specification describe the invention presented and the terms that are employed in the claims draw their meaning from the use of such terms in the specification. The same terms employed in the prior art may be broader in meaning than specifically employed herein. Whenever there is a question between the broader definition of such terms used in the prior art and the more specific use of the terms herein, the more specific meaning is meant.

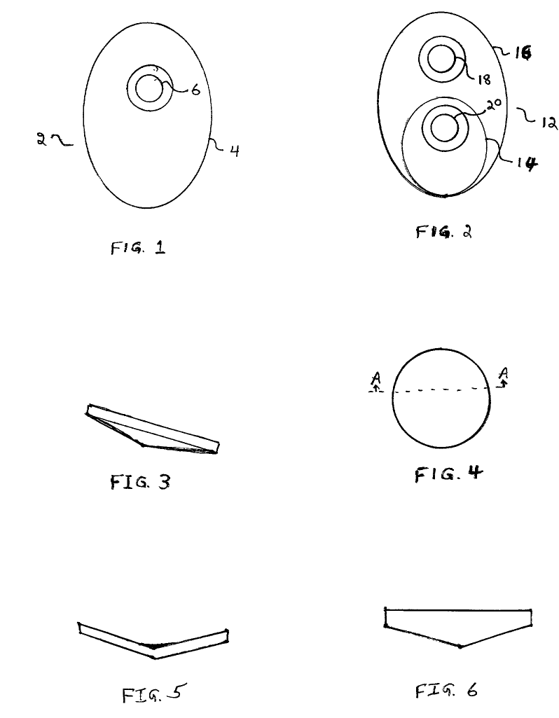

FIG. 1 illustrates a front view of a single-chambered breast implant. As seen in FIG. 1, breast prosthesis 2 is comprised of envelope 4 (also called shell 4), preferably comprising silicone sheet material. Envelope 4 embodies valve 6 formed as part of a wall to facilitate the introduction, containment, and removal of fluid within a chamber of envelope 4.

In one embodiment of the invention, at least two layers of a self-sealing silicone form envelope 4 and cover the exterior of valve 6. The layers are constructed such that, upon changes in the volume of prosthesis 2, the two layers move at varying rates. This allows holes formed within the material of envelope 4 as a result of a needle stick to become misaligned after the needle is removed, thereby minimizing chances of seepage or future leakage.

A breast implant according to one embodiment of the invention is constructed to withstand greater pressure than conventional, non-adjustable implants. Preferably, the material comprising the layer or layers of envelope 4 are thicker than the layer of an envelope of a conventional non-adjustable implant in order to ensure that the implant does not have wrinkles, even at a low fill volume.

In an alternative embodiment, bitumen implant 12 contains chambers 14 and 16, which either are in fluid communication or are not in fluid communication. If chambers 14 and 16 are not in fluid communication, then the bitumen implant has two separate port/valves 18 and 20, thereby allowing for volume changes to the base and volume changes to the projection portion to be made independently and consequently reducing step effects seen prevalently in implants in women who have small amounts of breast tissue and in mastectomy patients who have no breast tissue to cover the implant.

In an embodiment where bitumen implant 12 has two chambers in fluid communication, envelope 14, which is the “projection” portion of the implant is preferably constructed of a thinner material than the “base” portion of the implant, thereby resulting in a larger increase in the protrusion portion of the implant compared with the based portion and consequently minimizing step effects from volume increases. The projection portion would expand at a slightly greater rate than the base portion of the implant.

FIG. 3 shows a side view of backing plate 20. Preferably, backing plate 20 is made of ultra high molecular weight polyethylene (UHMWP). Alternatively, backing plate 20 can be made of any biocompatible hard material that is nonmagnetic and that is able to be imaged using ultrasound technology.

FIG. 4 shows a top view of backing plate 20. FIG. 5 shows a sectional view of backing plate 20 taken along section lines A-A of FIG. 4. The material comprising backing plate 20 must be sufficiently hard to withstand repeated needle impacts, must provide a positive target that allows identification of the center of backing plate 20 when ultrasound imaging is used, and should have a shape that does not provide a good target image when backing plate 20 is out of proper alignment. Thickness of backing plate 20 should be about 1/32^{nd} inch and can be about 1/16^{th} inch.

Backing plate 20 is preferably about ½ inch in diameter, ⅛ inch in profile height, and 1/16^{th} inch in thickness. The back of backing plate 20 is designed to avoid a false-positive ultrasound reading should the implant be 180° out of alignment. Backing plate 20 has a concave side and a convex side. The proper alignment is for the concave side to be facing the source of ultrasound waves. When the convex side faces the source of ultrasound waves, the waves are scattered and no image is detected.

Using a ½ inch backing plate allows for approximately 12 degrees of variance from the center of the needle target “bulls-eye.” A ¾ inch backing plate allows for approximately 16 degrees of variance from the center of the “bulls-eye.” Base plate 20 can be ½ inch to ¾ inch in diameter, both with a ⅛ inch angles lip that directs the needle into valve 6. The maximum diameter of backing plate 20 is approximately ¾ inch.

Preferable self-sealing silicone is placed in a buffer zone measuring ¼ inch to ½ inch beyond the limits of valve 6. Thus, the safe area for a needle stick on the implant can be as large as 2 inches and no smaller than 1½ inches based on the selected sizes. Of course, selecting other sizes changes the safe area for a needle stick on the implant.

I claim:

1. An implantable breast tissue prosthesis assembly comprising:
an envelope which has a shell defining an inner fluid containable chamber and an outer surface;
a valve formed as a part of a wall in the envelope, the valve facilitating the introduction, containment, or removal of fluid within the chamber of the envelope; and
the valve having a backing plate comprising material imageable by ultrasound imaging and shaped to guide a needle into the valve.

2. The prosthesis according to claim 1, wherein the backing plate is nonmagnetic.

3. The prosthesis according to claim 1, wherein the backing plate has a shape that scatters ultrasound waves when the backing plate is approximately 180° out of alignment.

4. A method for determining whether a nonmetallic valve formed on an implantable breast tissue prosthesis assembly is in proper alignment for needle insertion, the valve having a nonmetallic backing plate that is capable of being detected with ultrasound technology, the method comprising the steps of:
(a) placing a breast tissue prosthesis in proximity to a source of ultrasound energy;
(b) detecting reflected ultrasound energy; and
(c) determining by the presence and strength of any reflected signal whether the valve is in proper alignment.
